(19) United States
(12) Patent Application Publication
(10) Pub. No.: US 2006/0110412 A1
(43) Pub. Date: May 25, 2006

(54) Vaccine composition

(76) Inventors: Pierre Michel Desmons, King of Prussia, PA (US); Jan Poolman, King of Prussia, PA (US); Ramon Faustino Barbera Morales, King of Prussia, PA (US); Francisco Jesus Dominguez Alvarez, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/529,064

(22) PCT Filed: Jun. 10, 2003
(86) PCT No.: PCT/EP03/06094

§ 371 Date: Aug. 2, 2005

(30) Foreign Application Priority Data
Jun. 13, 2002 (GB) 0213622.4

(51) Int. Cl. A61K 39/095

(52) U.S. Cl. 424/250.1

(57) ABSTRACT

The present invention relates to vaccine compositions for the effective prevention of neisserial, preferably meningococcal, disease. The vaccine of the invention comprise a multivalent meningococcal bled composition comprising at least one bleb with homologous bactericidal activity which is derived from a meningococcal strain with a serosubtype that is prevalent in a country of use, and at least one bleb with heterologous bacterial activity which is derived from a meningococcal strain which need not have a serosubtype that is prevalent in the country of use.

### FIELD OF THE INVENTION

The present invention relates to the field of neisserial vaccine compositions, their manufacture, and the use of such compositions in medicine. More particularly it relates to the field of novel multivalent meningococcal outer-membrane vesicle (or bleb) vaccines, and advantageous methods of rendering such vaccines more effective.

### BACKGROUND OF THE INVENTION

Neisseria meningitidis (meningococcus) is a Gram-negative bacterium frequently isolated from the human upper respiratory tract. It occasionally causes invasive bacterial diseases such as bacteremia and meningitis. The incidence of meningococcal disease shows geographical seasonal and annual differences (Schwartz, B., Moore, P. S., Broome, C. V.; Clin. Microbiol. Rev. 2 (Supplement), S18-S24, 1989). Most disease in temperate countries is due to strans of serogroup B and varies in incidence from 1-10/100,000/year total population sometimes reaching higher values (Kaczmarski, E. B. (1997), Commun. Dis. Rep. Rev. 7: R55-9, 1995; Scholten, R. J. P. M., Bijlmer, H. A., Poolman, J. T. et al. Clin. Infect. Dis. 16: 237-246, 1993; Cruz, C., Pavez, G., Aguilar, E., et al. Epidemiol. Infect. 105: 119-126, 1990). Age-specific incidences in the two high risk-groups, infants and teenagers, reach higher levels.

Epidemics dominated by serogroup A meningococci occur, mostly in central Africa, sometimes reaching levels up to 1000/100,000/year (Schwartz, B., Moore, P. S., Broome, C. V. Clin. Microbiol. Rev. 2 (Supplement), S18-S24, 1989). Nearly all cases of meningococcal disease as a whole are caused by serogroup A, B, C, W-135 and Y meningococci. A tetravalent A, C, W-135, Y capsular polysaccharide vaccine is available (Armand, J., Arminjon, F., Mynard, M. C., Lafaix, C., J. Biol. Stand. 10: 335-339, 1982).

The polysaccharide vaccines are currently being improved by way of chemically conjugating them to carrier proteins (Lieberman, J. M., Chiu, S. S., Wong, V. K., et al. JAMA 275: 1499-1503, 1996). A serogroup B vaccine is not available, since the B capsular polysaccharide is non-immunogenic, most likely because it shares structural similarity to host components (Wyle, F. A., Artenstein, M. S., Brandt, M. L. et al. J. Infect. Dis. 126: 514-522, 1972; Finne, J. M., Leinonen, M., Mäkelä, P. M. Lancet ii.: 355-357, 1983).

Therefore, for many years efforts have been focused on developing meningococcal outer membrane vesicle (or bleb) based vaccines (de Moraes, J. C., Perkins, B., Camargo, M. C. et al. Lancet 340: 1074-1078, 1992; Bjune, G., Hoiby, E. A. Gronnesby, J. K et al. 338: 1093-1096, 1991). Such vaccines have the advantage of including several integral outer-membrane proteins in a properly folded conformation which can elicit a protective immunological response when administered to a host. In addition, Neisserial strains (including N. meningitidis serogroup B—menB) excrete outer membrane blebs in sufficient quantities to allow their manufacture on an industrial scale. Alternatively, blebs may be prepared by known methods comprising a detergent extraction of the bacterial cells (EP 11243), which has the benefit of removing some endotoxin (lipo-polysaccharides—or LPS; also called lipo-oligosaccharide—or LOS) from the vaccine.

Such multicomponent outer-membrane protein vaccines derived from wild-type menB strains have demonstrated efficacies from 57%-85% in older children (>4 years) and adolescents and have become registered in Latin America Most of these efficacy trials were performed with menB OMVs (outer membrane vesicles) made via a detergent extraction process.

Many bacterial outer membrane components are present in these vaccines, such as PorA, PorB, Rmp, Opc, Opa, FrpB and the contribution of these components to the observed protection still needs further definition. Other bacterial outer membrane components have been defined (using animal or human antibodies) as potentially being relevant to the induction of protective immunity, such as TbpB, NspA (Martin, D., Cadieux, N., Hamel, J., Brodeux, B. R., J. Exp. Med. 185: 1173-1183, 1997; Lissolo, L., Maître-Wilmotte, C., Dumas, p. et al., Inf. Immun. 63: 884-890, 1995). The mechanism of protective immunity will involve antibody mediated bactericidal activity and opsonophagocytosis.

The frequency of Neisseria meningitidis infections has risen in the past few decades in many European countries. This has been attributed to increased transmission due to an increase in social activities (for instance swimming pools, theatres, etc.). It is no longer uncomumon to isolate Neisseria meiningitidis strains that are less sensitive or resistant to some of the standard antibiotics.

### SUMMARY OF THE INVENTION

The present invention relates to vaccine compositions for the effective prevention or treatment of neisserial, preferably meningococcal, disease. The vaccines of the invention comprise a multivalent meningococcal bleb composition comprising at least one bleb with homologous bactericidal activity which is derived from a meningococcal strain with a serosubtype (PorA immunotype) that is prevalent in a country of use, and at least one bleb with heterologous bactericidal activity which is derived from a meningococcal strain which need not have a serosubtype that is prevalent in the country of use.

### DESCRIPTION OF THE INVENTION

The subject matter of and information disclosed within the publications and patents or patent applications mentioned in this specification are incorporated by reference herein. It should be understood that the word “comprising” used herein may be substituted for the term “consisting of” in all instances whilst still remaining within the scope of the invention.

The present inventors have found a solution to the problem of currently available meningococcal bleb vaccines only providing satisfactory Serum Bactericidal Activity (SBA) against homologous strains (to the strain from which the blebs were derived) and not satisfactory SBAs against heterologous strains. Usually the blebs in the art are derived from strains prevalent in a particular country or region. Although homologous protection is reasonable, the risk of an unprotected heterologous strain quickly gaining in prevalence is high, particularly in young children.

The present inventors have found that particular multi-valent bleb vaccine compositions (i.e. compositions comprising at least 2 different blebs) can provide a host with satisfatory SBAs against homologous and heterologous strains of Neisseria (particularly meningococcus). Such vaccines are advantageous in that rather than providing an expensive bleb vaccine made with many different blebs derived from all/most meningococcal strains infecting individuals in a country, the vaccines of the invention provide a good compromise by minimising the number of blebs in a vaccine, whilst still providing good specific and general protection against prevalent strains and against mutation of these strains or the introduction of new serogroup B strains when cases from prevalent strains are reduced.

Therefore in one aspect the present invention provides a multivalent meningococcal bleb composition comprising at least one (e.g. 1, 2, 3, 4, 5, 6, or 7) bleb preparation with homologous bactericidal activity which is derived from a meningococcal strain with a serosubtype PorA immunotype) that is prevalent in a country of use, and at least one (e.g. 1, 2, 3, 4, 5, 6 or 7) bleb preparation with heterologous bactericidal activity which is derived from a meningococcal strain which need not have a serosubtype that is prevalent in the country of use.

By a “meningococcus strain with a serosubtype that is prevalent in a country of use” it is meant the bleb is derived from a meningococcal strain with a serosubtype which is most prevalent (or possibly second or third or fourth prevalent—particularly if 2 or 3 or 4 bleb preparations with homologous bactericidal activity are incorporated in the vaccine) in percentage terms amongst strains of all serosubtypes which cause meningococcal disease in the country (or region or continent)—i.e. strains isolated during laboratory-based active surveillance of meningococcal disease in a country, region or continent Preferably the serosubtype of such a bleb constitutes more than 1, 2, 3, 4, 5, 6, 7, 8, 9, 10, 15, 20, 30, 40, 50 or 60% of all serosubtypes which cause meningococcal disease in the country (or region or continent).

If one bleb preparation with homologous bactericidal activity is included in the composition it is preferred that it is derived from a strain with a subserotype which is most prevalent in the country (or region or continent), if two or three or four are included then it is preferred that the strains used cover the two or three or four (respectively) most prevalent subserotypes.

By a “meningococcal strain which need not have a serosubtype that is prevalent in the country of use” it is meant that the bleb can be (but not necessarily be) derived from a meningococcal strain which is not from the most prevalent (or second, third, fourth, fifth or sixth) serosubtype in percentage terms amongst strains of all serosubtypes which cause meningococcal disease in the country (or region or continent)—i.e. strains isolated during laboratory-based active surveillance of sporadic cases of meningococcal disease in a country, region or continent. In such case, it is preferable that the serosubtype of such a bleb constitutes less than 1, 2, 3, 4, 5, 6, 7, 8, 9, 10, 15, 20, 30, 40, 50 or 60% of all serosubtypes which cause meningococcal disease in the country (or region or continent).

By “derived from a meningococcal strain” it is meant that the bleb is isolated from the meningococcal strain using any known method—such as a detergent-free isolation, or processes which involve detergent (such as deoxycholate) in the isolation.

By bleb preparations that have “homologous bactericidal activity” or “heterologous bactericidal activity” it is meant that the bleb preparations elicit satisfactory serum bactericidal activities (SBA) when administered to a host against homologous or heterologous meningococcal strains, respectively.

SBA is the most commonly agreed immunological marker to estimate the efficacy of a meningococcal vaccine (erbins et al. J Infect Dis. 1998, 177:683-691). Satisfactory SBA can be acertained by any known method. Preferably a blood sample is taken prior to the first vaccination, two months after the second vaccination and one month after the third vaccination (three vaccinations in one year being a typical human primary vaccination schedule administered at, for instance, 0, 2 and 4 months, or 0, 1 and 6 months). Such human primary vaccination schedules can be carried out on infants under 1 year old (for instance at the same time as Hib vaccinations are carried out) or 2-4 year olds or adolescents may also be vaccinated to test SBA with such a primary vaccination schedule. A further blood sample may be taken 6 to 12 months after primary vaccination and one month after a booster dose, if applicable.

SBA will be satisfactory for a bleb preparation with homologous bactericidal activity if one month after the third vaccine dose (of the primary vaccination schedule) (in 2-4 year old humans or adolescents, but preferably in infants in the first year of life) the percentage of subjects with a four-fold increase in terms of SBA (antibody dilution) titre (compared with pre-vaccination. titre) against the strain of meningococcus from which the bleb was derived is greater than 30%, preferably greater than 40%, more preferably greater than 50%, and most preferably greater than 60% of the subjects.

Of course a bleb preparation with heterologous bactericidal activity can also constitute bleb preparation with homologous bactericidal activity if it can also elicit satisfactory SBA against the meningococcal strain from which it is derived.

SBA will be satisfactory for a bleb preparation with heterologous bactericidal activity if one month after the third vaccine dose (of the primary vaccination schedule) (in 2-4 year old humans or adolescents, but preferably in infants in the first year of life) the percentage of subjects with a four-fold increase in terms of SBA (antibody dilution) titre (compared with pre-vaccination titre) against three heterologous strains of meningococcus is greater than 20%, preferably greater than 30%, more preferably greater than 35%, and most preferably greater than 40% of the subjects. Such a test is a good indication of whether the bleb preparation with heterologous bactericidal activity can induce cross-bactericidal antibodies against various meningococcal strains. The three heterologous strains should preferably have different electrophoretic type (ET)-complex or multilocus sequence typing (NST) pattern (see Maiden et al. PNAS USA 1998, 95:3140-5) to each other and preferably to the strain from which the bleb preparation with heterologous bactericidal activity is made. A skilled person will readily be able to determine three strains with different ET-complex which reflect the genetic diversity observed amongst meningococci, particularly amongst meningococcus type B strains that are recognised as being the cause of significant disease burden and/or that represent recognised MenB hyper-virulent lineages (see Maiden et al. supra). For instance three strains that could be used are the following: BZ10 (B:2b:P1.2) belonging to the A-4 cluster, B16B6 (B:2a:P1.2) belonging to the ET-37 complex; and H44/76 (B:15:P1.7,16) belonging to the ET-5 complex, or any other strains belonging to the same ET/Cluster. Such strains may be used for testing a bleb preparation with heterologous bactericidal activity made from, for instance, meningococcal strain CU385 (B3:4:P1.15) which belongs to the ET-5 complex. Another sample strain that could be used (for instance instead of any of the 3 test strains mentioned above) is from the Lineage 3 epidemic clone (e.g. NZ124 [B:4:P1.7,4]). Another ET-37 strain that could be used interchangeably with B16B6 is NGP165 (13:2a:P1.2).

Processes for measuring SBA activity are known in the art. For instance a method that might be used is described in WO 99/09176 in Example 10C. In general terms, a culture of the strain to be tested is grown (preferably in conditions of iron depletion—by addition of an iron chelator such as EDDA to the growth medium) in the log phase of growth. This can be suspended in a medium with BSA (such as Hanks medium with 0.3% BSA) in order to obtain a working cell suspension adjusted to approximately 20000 CFU/ml. A series of reaction mixes can be made mixing a series of two-fold dilutions of sera to be tested (preferably heat-inactivated at 56° C. for 30 min) [for example in a 50 μl/well volume) and the 20000 CFU/ml meningococcal, strain suspension to be tested [for example in a 25 μl/well volume]. The reaction vials should be incubated (e.g. 37° C. for 15 minutes) and shaken (e.g. at 210 rpm). The final reaction mixture [for example in a 100 μl volume] additionally contains a complement source [such as 25% final volume of pretested baby rabbit serum], and is incubated as above [e.g. 37° C. for 60 min]. For human SBA serology, human serum is usually used as the source of complement rather than baby rabbit complement, and the buffer used is PBS MgCl₂ 0.5 mM CaCl₂ 0.9 mM glucose 0.1% pH 7.4. A sterile polystyrene U-bottom 96-well microtiter plate can be used for this assay. A aliquot [e.g. 10 μl] can be taken from each well using a multichannel pipette, and dropped onto Mueller-Hinton agar plates (preferably containing 1% Isovitalex and 1% heat-inactivated Horse Serum) and incubated (for example for 18 hours at 37° C. in 5% CO₂). Preferably, individual colonies can be counted up to 80 CFU per aliquot. The following three test samples can be used as controls: buffer+bacteria+complement; buffer+bacteria+inactivated complement; serum+bacteria+inactivated complement. SBA titers can be straightforwardly calculated using a program which processes the data to give a measurement of the dilution which corresponds to 50% of cell killing by a regression calculation.

In a further aspect of the invention the present inventors have found that the bleb preparation with heterologous bactericidal activity of the invention can achieve its cross-bactericidal properties by being deficient in immunodominant outer membrane proteins (OMPs) compared to normal wild-type bleb preparations.

Thus the present invention also provides a multivalent meningococcal bleb composition of the invention wherein the bleb with heterologous bactericidal activity is deficient in an immunodominant outer membrane protein compared to blebs derived from a comparator strain (for instance wild-type MC58, but preferably wild-type strain H44/76), and the bleb with homologous bactericidal activity is not deficient to the same degree (or preferably at all) in said immunodominant outer membrane protein compared to blebs derived from the same comparator strain.

A skilled person will readily understand what is an immunodominant OMP, but it is preferred that the immunodominant OMP of the invention has highly immunogenic, surface-exposed epitopes (preferably within surface-exposed loop sequences) and is one of the top 10 highly-expressed OMPs (either by weight or by No. of molecules per cell) on the meningococcal surface. Usually these OMPs are highly immunogenic, but are quite variable in the amino acid sequence of their loop structures from strain to strain. Examples of such OMPs are detailed below.

By “deficient” it is meant that the bleb preparation with heterologous bactericidal activity of the invention has less of the immunodominant OMP of the invention on its surface than blebs made from the comparator strain. In particular the bleb preparation with heterologous bactericidal activity should have less than 98, 95, 90, 80, 70, 60, 50, 40, 30, 20, 10, or 5% of the amount of the immunodominant OMP as compared to the same quantity of bleb (as measured by total protein, usually around 10 μg of protein) prepared from the comparator strain. This can be assessed, for instance, by OMP band densitometry on an SDS-PAGE gel, for example as described in Example 4. Most preferably the bleb preparation with heterologous bactericidal activity of the invention should have none of the immunodominant OMP. The above is also the definition of “deficient” where this document compares the level of immunodominant OMP on the surface of the bleb production strain with respect to the comparator strain. A bleb or strain may, optionally, also be deficient in the immunodominant OMP if the OMP is engineered not to be surface-exposed on the outer membrane of the bleb/strain, or if the OMP is expressed at the same level but one or more surface-exposed loops are engineered to be less variable or immunodominant by their replacement, mutation or deletion in order to give blebs made from such a strain more heterologous bactericidal activity.

Preferably the process of deriving the bleb preparation with heterologous bactericidal activity from the source meningococcal strain is the same as that used to derive the blebs from the comparator strain (preferably the method described in Frederiksen et al. NIPH Annals. 1991, 14:67-80). This need not be the case, however, particularly if the reason for the bleb preparation with heterologous bactericidal activity being deficient in the immunodominant OMP is due to the way that the blebs are produced. In such case the standard procedure for bleb production for the comparator strain is the method as described in Frederiksen et al. (NIPH Annals. 1991, 14:67-80) or Bjune et al. (NIPH Annals 1991 14:81-93).

The bleb with heterologous bactericidal activity may be derived from a wild-type meningococcal strain that is naturally deficient in the immunodominant outer membrane protein, or may be derived from meningococcal strains rendered deficient in the immunodominant outer membrane protein.

In particular, the bleb with heterologous bactericidal activity can be rendered deficient in the immunodominant outer membrane protein by genetically-engineering the production strain to be deficient in the OMP or to produce less or none of the OMP compared to the wild-type strain from which it is engineered.

By “less or none” it is meant in particular that the strain expresses less than 98, 95, 90, 80, 70, 60, 50, 40, 30, 20, 10, or 5% of the amount of the immunodominant OMP on its surface as compared to the wild-type strain from which it was engineered. Most preferably the engineered strain does not express any of the immunodominant OMP. A strain may also have less or no immunodominant OMP if the OMP is engineered not to be surface-exposed on the outer membrane of the strain, or, optionally, if the OMP is expressed at the same level but one or more surface-exposed loops are engineered to be less variable or immunodominant by their replacement, mutation or deletion in order to give blebs made from such a strain more heterologous bactericidal activity.

The gene encoding the immunodominant OMP of the invention may be engineered in the above way by known techniques. In particular the meningococcal strain may be genetically altered in either the promoter or coding region of the gene such that the strain produces less or none of said immunodominant outer membrane protein. Particular ways that this may be achieved are described in WO 01/09350. For instance a transposon (or other sequence) may be inserted to disrupt the coding region or promoter region of the gene, or point mutations or deletions may achieve a similar result. The promoter or coding region may be deleted completely or in part in order to render the product of the gene less immunodominant (for instance by replacing, mutating, or deleting immunogenic epitopes present in the surface-exposed loops). Recombination events can be used to delete, insert, replace or mutate sequences in the OMP to render them less immunodominant such as the replacement of a strong promoter for a weaker (or no) promoter. Frameshift mutations may also be inserted into the coding region.

Without wishing to be bound by theory, it is thought that the combination of such a bleb with a bleb with homologous bactericidal activity is effective as a vaccine, as the bleb with homologous bactericidal activity is effective against prevalent strains in a country of use by virtue of bactericidal antibodies being generated against the immunodominant (but variable) OMP of the invention, however because the immunodominant OMP of the invention can immunologically mask the efficacy of more conserved antigens present on the bleb surface (which are present at lower levels), the bleb with homologous bactericidal activity does not have satisfactory heterologous bactericidal activity, with the above disadvantages. The bleb with heterologous bactericidal activity of the invention has this masking removed to some degree so that the conserved OMPs present at low level in the blebs have more influence on the host's immune system, and can thus elicit cross-bactericidal antibodies in a host with satisfactory heterologous bactericidal activity. The combination of both types of bleb provides a preparation that can be formulated into an optimal vaccine for use in a particular country or region.

Preferably the bleb with heterologous bactericidal activity is deficient in (or has been engineered to have less or none of) an immunodominant OMP which is one or more of the following antigens: PorA, PorB, OpC, OpA or PIuC. Preferably the bleb is deficient in (or has been engineered to have less or none of) PorA.

A preferred bleb preparation with heterologous bactericidal activity of the invention that is naturally deficient in PorA is a bleb preparation isolated from meningococcal strain CU-385 (B:4:P1.19,15), preferably isolated by the method disclosed in EP 301992-B. PorA is deficient in this strain or bleb compared to the comparator strain or bleb (e.g. H44/76). Bleb preparations from this strain (or blebs from similar strains with equivalent or lower levels of PorA [e.g. less than 25, 22, 20, 15, 10 or 5% PorA of total bleb protein] as compared with CU-385 or CU-385 blebs) have been found to be well-suited to be combined with bleb preparations with homologous bactericidal activity with normal (equivalent or greater—e.g. more than 28, 30, 35, or 40% PorA of total bleb protein) levels of PorA as compared with H44/76 blebs. Such bleb combinations (particularly those comprising CU-385 blebs) are thus preferred bleb combinations of the present invention. Preferably PorA quantities are assessed in the final bleb preparations.

Furthermore, the bleb with heterologous bactericidal activity of the invention may be further improved in terms of its cross-bactericidal (satisfactory SBA) properties. This may be achieved by enhancing the quantity of certain OMs on the surface of said bleb.

Accordingly, the bleb with heterologous bactericidal activity of the invention as hereinbefore described is preferably derived from an engineered meningococcal strain which has upregulated expression of one or more of the following genes (either by adding an extra copy of the gene to the strain, or by inserting a stronger promoter upstream of the existing gene, or any other way described in WO 01/09350): NspA (WO 96/29412), Hsf-like or truncates thereof (WO 99/31132 & WO 01/55182; also known as NhhA), Hap (PCT/EP99/02766), OMP85 (WO 00/23595), PilQ (PCT/EP99/03603), PIDA (PCT/EP99/06718), FrpB (WO 96/31618), ThpA (WO92/03467, U.S. Pat. No. 5,912,336, WO93/06861 and EP586266), TbpB (WO93/06861 and EP586266), NadA (Comanducci et al J. Exp. Med. 2002 195; 1445-1454), FrpA and/or FrpC (WO 92/01460; Thompson et al., (1993) J. Bacteriol. 175:811-818; Thompson et al., (1993) Infect. Immun. 61:2906-2911), LbpA, LbpB (PCT/EP98/05117), FhaB (WO98/02547, SEQ ID NO 38 [nucleotides 3083-9025]), HasR (PCT/EP99/05989), lipo02 (PCT/EP99/08315), Thp2 (WO 99/57280), MltA (WO 99/57280), TspA (WO 00/03003), TspB (WO 00/03003) and ctrA (PCT/EP00/00135).

“Upregulated expression” refers to any means to enhance the expression of an antigen of interest, relative to that of the non-modified (i.e., naturally occurring) bleb or strain. It is understood that the amount of ‘upregulation’ will vary depending on the particular antigen of interest but will not exceed an amount that will disrupt the membrane integrity of the bleb. Upregulation of an antigen refers to expression that is at least 10% higher than that of the non-modified bleb or strain. Preferably it is at least 50% higher. More preferably it is at least 100% (2, 3, 5 or 10 fold) higher.

It should be understood that multivalent bleb preparations (or vaccines comprising such preparations) of the prior art which may have the characteristics of the multivalent bleb preparations of the present invention are not claimed; for instance any particular multivalent bleb preparations disclosed in WO 01/09350 (such as a combination consisting of blebs derived from all of the following menB strains: H44/76, M97/252078, BZ10, NGP165 and CU385, or combinations consisting of blebs from CU3 85 and one or more bleb preparations derived from one or more of the other 4 strains) are not claimed, nor are any particular multivalent bleb preparations disclosed in WO 02/09643 (such as a combination consisting of blebs derived from all of the following meningococcal strains: MenC RM1090, MenB BZ198, MenA Z1092, or combinations consisting of a pair of bleb preparations derived from any pair of these 3 strains), nor are any particular multivalent bleb preparations disclosed in WO 01/91788.

Vaccine Formulations

A preferred embodiment of the invention is the formulation of the multivalent bleb compositions of the invention in a vaccine for the treatment or prevention of neisserial (preferably meningococcal) disease which may also comprise a pharmaceutically acceptable excipient.

The manufacture of bleb preparations from any of the aforementioned strains (unless otherwise stated) may be achieved by any of the methods well known to a skilled person. Preferably the methods disclosed in EP 301992, U.S. Pat. No. 5,597,572, EP 11243 or U.S. Pat. No. 4,271,147, Frederikson et al. (NIPH Annals [1991], 14:67-80), Zollinger et al. (J. Clin. Invest. [1979], 63:836-848), Saunders et al. (Infect. Immun. [1999], 67:113-119), Drabick et al. (Vaccine [2000], 18:160-172) or WO 01/09350 (Example 8) are used. In general, OMVs are extracted with a detergent, preferably deoxycholate, and nucleic acids are optionally removed enzymatically. Purification is achieved by ultracentrifligation optionally followed by size exclusion chromatography. The 2 or more different blebs of the invention may be combined in a single container to form a multivalent preparation of the invention (although a preparation is also considered multivalent if the different blebs of the invention are separate compositions in separate containers which are administered at the same time [the same visit to a practitioner] to a host). OMV preparations are usually sterilised by filtration through a 0.2 μm filter, and are preferably stored in a sucrose solution (e.g. 3%) which is known to stabilise the bleb preparations.

It should be noted that the blebs constituting the multivalent bleb preparations of the invention are derived from genetically diverse strains (i.e. a comparison of all the bleb-producing strains used to make the preparations of the invention shows that more than 3% of the open-reading frames of the genome of any given strain have genetic differences with the respective open-reading frames of the genomes of the other strans used). Most preferably, however, the multivalent bleb preparations of the invention are derived entirely from meningococcus B strains.

Vaccine preparation is generally described in Vaccine Design (“The subunit and adjuvant approach” (eds Powell M. F. & Newman M. J.) (1995) Plenum Press New York).

The multivalent bleb compositions of the present invention may be adjuvanted in the vaccine formulation of the invention. Suitable adjuvants include an aluminium salt such as aluminum hydroxide gel, alum, or aluminium phosphate (preferably aluminium hydroxide), but may also be a salt of calcium (particularly calcium carbonate), iron or zinc, or may be an insoluble suspension of acylated tyrosine, or acylated sugars, cationically or anionically derivatised polysaccharides, or polyphosphazenes.

Suitable Th1 adjuvant systems that may be used include, Monophosphoryl lipid A, particularly 3-de-O-acylated monophosphoryl lipid A (or other non-toxic is derivatives of LPS), and a combination of monophosphoryl lipid A, preferably 3-de-O-acylated monophosphoryl lipid A (3D-MPL) [or non toxic LPS derivatives] together with an aluminium salt. An enhanced system involves the combination of a monophosphoryl lipid A and a saponin derivative particularly the combination of QS21 [or other saponin] and 3D-MPL [or non toxic LPS derivative) as disclosed in WO 94/00153, or a less reactogenic composition where the QS21 [or saponin] is quenched with cholesterol as disclosed in WO96/33739. A particularly potent adjuvant formulation involving QS21, 3D-MPL and tocopherol in an oil in water emulsion is described in WO95/17210 and is a preferred formulation.

The vaccine may comprise a saponin, more preferably QS21. It may also comprise an oil in water emulsion and tocopherol. Umethylated CpG containing oligo nucleotides (WO 96/02555) are also preferential inducers of a TH1 response and are suitable for use in the present invention.

The vaccine preparation of the present invention may be used to protect or treat a mammal susceptible to infection, by means of administering said vaccine via 30 systemic or mucosal route. These administrations may include injection via the intramuscular, intraperitoneal, intradermal or subcutaneous routes; or via mucosal administration to the oral/alimentary, respiratory, genitourinary tracts. Thus one aspect of the present invention is a method of immunizing a human host against a disease caused by neisserial (preferably meningococcal) bacteria, which method comprises administering to the host an immunoprotective dose of the multivalent bleb preparation of the present invention.

The amount of bleb in each vaccine dose is selected as an amount which induces an immunoprotective response without significant, adverse side effects in typical vaccinees. Such amount will vary depending upon which specific immunogen is employed and how it is presented. Generally, it is expected that each dose will comprise 1-100 μg of each bleb (in terms of protein), preferably 5-50 μg, and most typically in the range 5-25 μg. Therefore for a bivalent bleb vaccine of the invention each dose may typically include 2×25 μg of bleb.

An optimal amount of each bleb in a particular vaccine can be ascertained by standard studies involving observation of appropriate immune responses in subjects. Following an initial (primary) vaccination (typically 3 administrations, preferably in the first year of life or within a single year during adolescence, for instance at 0, 2 and 4 months, or 0, 1 and 6 months), subjects may receive one or several booster immunisations (after 1 year following the first primary administration) adequately spaced. The vaccine of the invention may be used to immunize babies in the first year of life, 2-4 year olds, or adolescents. It is particularly advantageous in eliciting satisfactory heterologous bactericidal activity in babies in the first year of life.

Ghost or Killed Whole Cell Vaccines

The inventors envisage that the above improvements to multivalent bleb preparations and vaccines can be easily extended to ghost or killed whole cell preparations and vaccines (with identical advantages). The meningococcal strains used to make the multivalent bleb preparations of the invention can also be used to made multivalent ghost and killed whole cell preparations. Methods of making ghost preparations (empty cells with intact envelopes) from Gram-negative strains are well known in the art (see for example WO 92/01791). Methods of killing whole cells to make inactivated cell preparations for use in vaccines are also well known. The terms ‘bleb preparations’ and ‘bleb vaccines’ as well as the processes described throughout this document are therefore applicable to the terms ‘ghost preparation’ and ‘ghost vaccine’, and ‘killed whole cell preparation’ and ‘killed whole cell vaccine’, respectively, for the purposes of this invention.

Preferred Vaccine Compositions of the Invention

A preferred vaccine of the invention as described above which is particularly suitable for use in a vaccination program in New Zealand or Europe (preferably the European Union) comprises a multivalent bleb composition of the invention where the bleb with homologous bactericidal activity is derived from a meningococcal strain with a serosubtype of P1.4.

A preferred vaccine of the invention as described above which is particularly suitable for use in a vaccination program in USA comprises a multivalent bleb composition of the invention where the bleb with homologous bactericidal activity is derived from a meningococcal strain with a serosubtype of P1.7,16, and optionally further blebs with homologous bactericidal activity are also included derived from one or more (2, 3 or all 4) meningococcal strains with serosubtypes selected from the following list: P1.7,1 ; P1.5,2 ; P1.22a,14; and P1.14.

A preferred vaccine of the invention as described above which is particularly suitable for use in a vaccination program in Norway comprises a multivalent bleb composition of the invention where the bleb with homologous bactericidal activity is derived from a meningococcal strain with a serosubtype of P1.16.

For all the above vaccines, the bleb with heterologous bactericidal activity that the vaccine comprises is preferably derived from strain CU-385 (or similar strains or bleb preparations which have equivalent or less PorA than CU-385 or CU-385 blebs, respectively, as described herein). As described above, it is then further preferred that the above-listed bleb with homologous bactericidal activity or the strain from which it is derived has equivalent or more PorA than H44/76 bleb, or strain, respectively. Preferably PorA quantities are assessed in the final bleb preparations.

Vaccine Combinations

A further aspect of the invention is vaccine combinations comprising the multivalent bleb preparations or vaccines of the invention with other antigens which are advantageously used against certain disease states. It has been found that blebs are particularly suitable for formulating with other antigens, as they advantageously have an adjuvant effect on the antigens they are mixed with.

In one preferred combination, the multivalent meningoccocal bleb preparations or vaccines of the invention are formulated with 1, 2, 3 or preferably all 4 of the following meningococcal capsular polysaccharides which may be plain or conjugated to a carrier comprising T-cell epitopes (preferably a protein carrier such as tetanus toxoid, diptheria toxoid, or CRM197): A, C, Y or W. The term “polysaccharide” is intended to cover unsized or sized polysaccharides, or sized oligosaccharides. Preferably at least C and Y are included in a European vaccine, at least C is included in a US vaccine, and at least A and C are included in a vaccine for an African, South American, or equatorial country. Such a vaccine may be advantageously used as a global meningococcus vaccine.

In a further preferred embodiment, the multivalent bleb preparations or vaccines of the invention (preferably formulated with 1, 2, 3 or all 4 of the plain or conjugated meningococcal capsular polysaccharides A, C, Y or W) are formulated with a conjugated H. influenzae b capsular polysaccharide, and/or one or more plain or conjugated pneumococcal capsular polysaccharides. Optionally, the vaccine may also comprise one or more protein antigens that can protect a host against Streptococcus pneumoniae infection. Such a vaccine may be advantageously used as a global meningitis vaccine.

The pneumococcal capsular polysaccharide antigens are preferably selected from serotypes 1, 2, 3, 4, 5, 6B, 7F, 8, 9N, 9V, 10A, 11A, 12F, 14, 15B, 17F, 18C, 19A, 19F, 20, 22F, 23F and 33F (most preferably from serotypes 1, 3, 4, 5, 6B, 7F, 9V, 14, 18C, 19F and 23F).

Preferred pneumococcal proteins antigens are those pneumococcal proteins which are exposed on the outer surface of the pneumococcus (capable of being recognised by a host's immune system during at least part of the life cycle of the pneumococcus), or are proteins which are secreted or released by the pneumococcus. Most preferably, the protein is a toxin, adhesin, 2-component signal tranducer, or lipoprotein of Streptococcus pneumoniae, or truncate or immunologically functional equivalent thereof. Particularly preferred proteins include, but are not limited to: pneumolysin (preferably detoxified by chemical treatment or mutation) [Mitchell et al. Nucleic Acids Res. Jul. 11, 1990; 18(13): 4010 “Comparison of pneumolysin genes and proteins from Streptococcus pneumnoniae types 1 and 2.”, Mitchell et al. Biochim Biophys Acta Jan. 23, 1989; 1007(1): 67-72 “Expression of the pneumolysin gene in Escherichia coli: rapid purification and biological properties.”, WO 96/05859 (A. Cyanamid), WO 90/06951 (Paton et al), WO 99/03884 (NAVA)]; PspA and transmembrane deletion variants thereof (U.S. Pat. No. 5,804,193—Briles et al.); PspC and transmembrane deletion variants thereof (WO 97/09994—Briles et al); PsaA and transmembrane deletion variants thereof (Berry & Paton, Infect Immun December 1996; 64(12):5255-62 “Sequence heterogeneity of PsaA, a 37-kilodalton putative adhesin essential for virulence of Streptococcus pneumoniae”); pneumococcal choline binding proteins and transmembrane deletion variants thereof; CbpA and transmembrane deletion variants thereof (WO 97/41151; WO 99/51266); Glyceraldehyde-3-phosphate—dehydrogenase (Infect. Immun. 1996 64:3544); HSP70 (WO 96/40928); PcpA (Sanchez-Beato et al. FEMS Microbiol Lett 1998, 164:207-14); M like protein, SB patent application No. EP 0837130; and adhesin 18627, SB Patent application No. EP 0834568. Further preferred pneumococcal protein antigens are those disclosed in WO 98/18931, particularly those selected in WO 98/18930 and PCT/US99/30390.

Further preferred Streptococcus pneumoniae protein antigens of the invention are selected from the group consisting of: Poly Histidine Triad family (Pht; in particular PhtA, PhtB, PhtD, or PhtE), Lyt family (in particular LytA, LytB, or LytC), SpsA, Sp128, Sp130, Sp125, Sp101 and Sp133, or truncate or immunologically functional equivalent thereof.

For the purposes of this invention, “immunologically functional equivalent” is defined as a peptide of protein comprising at least one protective epitope from the proteins of the invention. Such epitopes are characteristically surface-exposed, highly conserved, and can elicit a bactericidal antibody response in a host or prevent toxic effects. Preferably, the functional equivalent has at least 15 and preferably 30 or more contiguous amino acids from the protein of the invention. Most preferably, fragments, deletions of the protein, such as transmembrane deletion variants thereof (ie the use of the extracellular domain of the proteins), fusions, chemically or genetically detoxified derivatives and the like can be used with the proviso that they are capable of raising substantially the same immune response as the native protein. The position of potential B-cell epitopes in a protein sequence may be readily determined by identifying peptides that are both surface-exposed and antigenic using a combination of two methods: 2D-structure prediction and antigenic index prediction. The 2D-structure prediction can be made using the PSIPRED program (from David Jones, Brunel Bioinformatics Group, Dept. Biological Sciences, Brunel University, Uxbridge UB8 3PH, UK). The antigenic index can be calculated on the basis of the method described by Jameson and Wolf (CABIOS 4:181-186 [1988]).

The Streptococcus pneumoniae protein of the invention is preferably selected from the group consisting of: a protein from the polyhistidine triad family (Pht), a protein from the Lyt family, a choline binding protein, proteins having an LPXTG motif (where X is any amino acid), proteins having a Type II Signal sequence motif of LXXC (where X is any amino acid), and proteins having a Type I Signal sequence motif. Preferred examples within these categories (or motifs) are the following proteins (or truncate or immunologically functional equivalent thereof):

The Pht (Poly Histidine Triad) family comprises proteins PhtA, PhtB, PhtD, and PhtE. The family is characterised by a lipidation sequence, two domains separated by a proline-rich region and several histidine triads, possibly involved in metal or nucleoside binding or enzymatic activity, (3-5) coiled-coil regions, a conserved N-terminus and a heterogeneous C terminus. It is present in all strains of pneumococci tested. Homologous proteins have also been found in other Streptococci and Neisseria. Preferred members of the family comprise PhtA, PhtB and PhtD. More preferably, it comprises PhtA or PhtD. It is understood, however, that the terms Pht A, B, D, and E refer to proteins having sequences disclosed in the citations below as well as naturally-occurring (and man-made) variants thereof that have a sequence homology that is at least 90% identical to the referenced proteins. Preferably it is at least 95% identical and most preferably it is 97% identical.

With regards to the Pht proteins, PhtA is disclosed in WO 98/18930, and is also referred to Sp36. As noted above, it is a protein from the polyhistidine triad family and has the type II signal motif of LXXC.

PhtD is disclosed in WO 00/37105, and is also referred to Sp036D. As noted above, it also is a protein from the polyhistidine triad family and has the type II LXXC signal motif.

PhtB is disclosed in WO 00/37105, and is also referred to Sp036B. Another member of the PhtB family is the C3-Degrading Polypeptide, as disclosed in WO 00/17370. This protein also is from the polyhistidine triad family and has the type II LXXC signal motif. A preferred immunologically functional equivalent is the protein Sp42 disclosed in WO 98/18930. A Pht truncate (approximately 79 kD) is disclosed in WO99/15675 which is also considered a member of the PhtX family.

PhtE is disclosed in WO00/30299 and is referred to as BVH-3.

SpsA is a Choline binding protein (Cbp) disclosed in WO 98/39450.

The Lyt family is membrane associated proteins associated with cell lysis. The N-terminal domain comprises choline binding domain(s), however the Lyt family does not have all the features found in the choline binding protein family (Cbp) family noted below and thus for the present invention, the Lyt family is considered distinct from the Cbp family. In contrast with the Cbp family, the C-terminal domain contains the catalytic domain of the Lyt protein family. The family comprises LytA, B and C. With regards to the Lyt family, LytA is disclosed in Ronda et al., Eur J Biochem, 164:621-624 (1987). LytB is disclosed in WO 98/18930, and is also referred to as Sp46. LytC is also disclosed in WO 98/18930, and is also referred to as Sp91. A preferred member of that family is LytC.

Another preferred embodiment are Lyt family truncates wherein “Lyt” is defined above and “trincates” refers to proteins lacking 50% or more of the Choline binding region. Preferably such proteins lack the entire choline binding region.

Sp125 is an example of a pneumococcal surface protein with the Cell Wall Anchored motif of LPXTG (where X is any amino acid). Any protein within this class of pneumococcal surface protein with this motif has been found to be useful within the context of this invention, and is therefore considered a further protein of the invention. Sp125 itself is disclosed in WO 98/18930, and is also known as ZmpB—a zinc metalloproteinase.

Sp101 is disclosed in WO 98/06734 (where it has the reference #y85993. It is characterised by a Type I signal sequence.

Sp133 is disclosed in WO 98/06734 (where it has the reference #y85992. It is also characterised by a Type I signal sequence.

Sp128 and Sp130 are disclosed in WO 00/76540.

The proteins used in the present invention are preferably selected from the group PhtD and PhtA, or a combination of both of these proteins, or a combination or either or both with CbpA.

Further Aspects of the Invention

Further aspects of the invention provide: a method of manufacturing the multivalent meningococcal bleb composition or the vaccine of the invention comprising the step of combining a bleb with homologous bactericidal activity with a bleb with heterologous bactericidal activity; a method of preventing or treating neisserial, preferably meningococcal, disease comprising the step of administering an immunologically effective amount of the vaccine of the invention (usually with a 3 dose primary immunisation scheme, preferably where each immunisation is separated by 1-2 months, and optionally boosted) to a host in need thereof (preferably a 2-4 year old or adolescent human, and advantageously for an infant of less than two [preferably one] years); and the use of an immunologically effective amount of the vaccine of the invention in the manufacuture of a medicament for the prevention or treatment of neisserial, preferably meningococcal, disease (particularly where the prevention of treatment is via a a 3 dose primary immunisation scheme [preferably where each immunisation is separated by 1-2 months, and optionally boosted] and/or the prevention or treatment of disease is in a human—preferably a 2-4 year old or adolescent, and advantageously an infant of less than two [preferably one] years).

### EXAMPLES

The examples below are carried out using standard techniques, which are well known and routine to those of skill in the art, except where otherwise described in detail. The examples are illustrative, but do not limit the invention.

### Example 1

### Construction of a Neisseiria meningdtidis Serogoup B Strain Lacking the Major Immunodominant Antigen PorA

This is described in Example 3 of WO 01/09350.

### Example 2

### Benefits of the Multivalent Meningococcal Bleb Vaccines of the Invention

A number of efficacy trials have been performed with serogroup B OMV vaccines. Two specific OMv vaccines were developed to combat epidemic situations in Cuba and Norway due mostly to single serosubtypes, i.e. 4:P1.15 and 15:P1.16, respectively. Both efficacy trials have been performed in teenagers in a placebo-controlled double-blind fashion. In Cuba an efficacy of 83% (confidence limits: 42%-95%) was found with a follow-up time of 16 months (Sierra NIPH Annals 1991, 14:195-207) whilst in Norway an efficacy of 57% was found with a follow-up time of 29 months (lower confidence limit: 27%) Bjune et al. NIPH Annals 1991 14:81-93). In Norway the efficacy was 86% at a follow-up time of 10 months, and a later third dose demonstrated enhanced immunogenicity and improved antibody persistence Rosenquist, Infenct. Immun. 1995 63:4642-4652). Since both trials were performed in a mostly type-homologous setting, no conclusions could be made about cross-protection.

When compared to the Norwegian vaccine in two head-to-head trials and using cross-bactericidal activity against the respective strains, the Cuban vaccine appears to induce a higher level of cross-bactericidal activity (see Table 1).

TABLE 1
Serum Bactericidal Activity (SBA) induced by the Finlay meningoccocal
BC vaccine - % of SBA responders (Post dose 3)
Icelandic trial (Perkins et al, JID, 177, 1998, 683-691)

| | | | | | 4: P1.15 | |
|---|---|---|---|---|---|---|
| | | | | 15: P1.16 | (Cuban | 4: P1.4 |
| Age | | | N | (Norwegian strain) | strain) | (NZ type) |
| Finlay | 16-19 | yrs | 74 | 48 | 44 | 36 |
| NIPH | | | 75 | 84 | 31 | 27 |

Chilean trial (Tappero, JAMA, 281, 1999, 1520-27)

| | | | | 15: P1.16 | 4: P1.15 |
|---|---|---|---|---|---|
| Finlay | <1 | yr | 51 | 31 | 90 |
| | 2-4 | yrs | 48 | 41 | 78 |
| | 17-30 | yrs | 53 | 56 | 67 |
| NIPH | <1 | yr | 50 | 98 | 2 |
| | 2-4 | yrs | 51 | 98 | 24 |
| | 17-30 | yrs | 50 | 96 | 46 |

An SBA responder was defined as a person with a 4-fold or greater rise in SBA titre compared with pre-vaccination titre.
Infants and children were given three doses of Finlay or control (Hib) vaccine two months apart.
In adults, the two first doses were given 2 month apart. The 3^{rd} dose was given 2 months after the 2^{nd} dose in the Chilean study and one year after the 2^{nd} dose in the Icelandic trial.
Blood samples were taken prior to vaccination and 4-6 weeks after the second and third vaccination

The present inventors have determined that when comparing bactericidal activity, the Norwegian vaccine leads to a more strain-specific response, whilst the Cuban vaccine induces more cross-bactericidal activity. Furthermore, the present inventors believe the Cuban bleb vaccine has such an activity because it is deficient in an immunodominant OMP, in particular PorA. Although deficient in PorA, sufficient is present for homologous bactericidal activity to be also demonstrated with the Cuban vaccine.

The present inventors have found that a multivalent vaccine comprising a bleb with homologous bactericidal activity (having a serosubtype prevalent in the country of use) and a bleb with heterologous bactericidal activity provides an optimal vaccine which protects against locally prevalent epidemic strains, yet also provides heterologous protection which lowers the chance of the emergence of new serogroup B strains that could occur after implementation of a national mass immunisation campaign when the epidemic strain will decrease in prevalence.

An optimal vaccine for Europe and/or New Zealand will incorporate a combination of two separate blebs: P1.4 (epidemic strain) and P1.15 (with heterologous bactericidal activity—for instance OMV derived from strain CU-385 used to prepare the commercialised Cuban VA-MENGOC-BC® vaccine). This combination aims to provide homologous P1.4 protection as well retaining the heterologous protection of the Cuban P1.15 strain.

### Example 3

### Multivalent Bleb Vaccine Comprising Blebs Derived from Meningococcal Strain CU-385 (B4:P1.19.15), and New Zealand Epidemic Menigococcal Strain (B:4:P1.7b,4)

The above multivalent vaccine was made (25 μg of each bleb in each human dose, adjuvanted with aluminium hydroxide).

The monovalent and bivalent bleb preparations were tested to examine whether any immune interference resulted in combining the blebs together.

The level of induced functional antibodies was evaluated by a bactericidal test performed in mice with pooled serum samples.

Briefly, groups of 10 BALB/c mice (6-8 weeks old) were injected twice with the equivalent of 10 μg of protein of the adsorbed monovalent or bivalent bulks on day 0 and day 21. Blood samples were taken on day 35. Determination of the bactericidal activity in the sera was based on the property of the antibodies to induce bacterial lysis by complement activation. After incubating the sera with viable meningococcus B (both the CU-385 and P1.4 New Zealand strains were tested), in the presence of rabbit complement, the number of colony forming units (CFU) in the serum samples was determined. The Bactericidal titre is the latest serum dilution that gives more than 50% of killing.

Based on the results, the bivalent vaccine is immunogenic and induces functional antibodies. There was no indication of immune interference when the two monobulks were mixed.

### Example 4

### Relative PorA Expression Level in N. meningitidis Strains

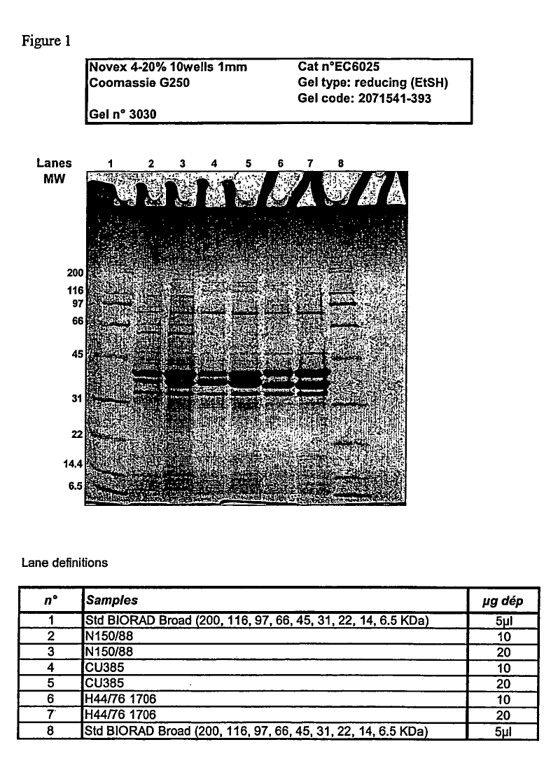

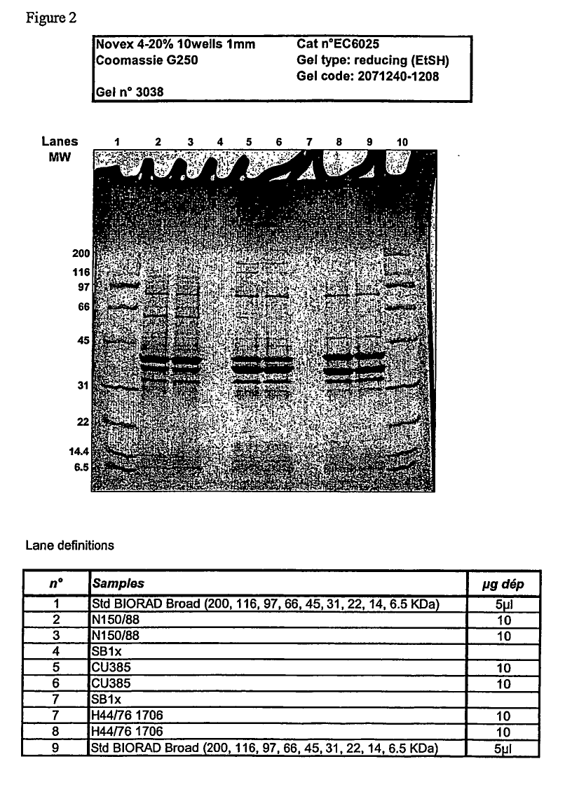

Outer-membrane vesicle samples (made essentially as described in NIPH Annals 1991 14:67-80) from strains CU385, H44/76 and N150/88 were compared by SDS-PAGE for PorA expression level. Samples were run on Novex 4-20% gels. In FIG. 1, 10 and 20 μg (Lowry determination) of materials were loaded per well, while in FIG. 2, 10 μg of materials were loaded in duplicates for each strain. After Coomassie Blue staining, 3 major bands were detected in the 3 strains: PorA (about 38 KDa) PorB (about 36 Kda) and RMP (about 33 Kda). The detected bands on lanes loaded with 10 μg of material were scanned with two different densitometers: Pharmacia Imagescanner densitometer, using 1D-Elite software, and Biorad desitometer, using MultiAnalist sofware. DO values were computed in order to determine the relative expression level of PorA for each strain. For each lane, the PorA level was expressed in % of the total of protein detected.

The results (table 1 below) show that the relative level of expression of PorA is lower in CU385 compared to the 2 other strains: PorA accounted for only about 20% of the total detected proteins in CU385 blebs, while it measured around 40 and 30% of protein in blebs from strains N150/88 and H44/76, respectively.

1. A multivalent meningococcal bleb composition comprising a bleb preparation deficient in PorA in that it has less than 80% of the amount of PorA as compared to the same quantity of blebs made from strain H44/76 and a bleb preparation that is not deficient in PorA compared to blebs made from strain H44/76.

2. The multivalent meningococcal bleb composition of claim 1, wherein the bleb preparation that is not deficient in PorA is derived from a meningococcal strain with a serosubtype that is prevalent in a country of use.

3. The multivalent meningococcal bleb composition of claim 1, wherein the bleb preparation deficient in PorA has less than 22% PorA of total bleb protein, or lacks PorA.

4. The multivalent meningococcal bleb composition of claim 1, wherein the bleb preparation not deficient in PorA has more than 28% PorA of total bleb protein.

5. The multivalent meningococcal bleb composition of claim 1, wherein the bleb preparation deficient in PorA is derived from the meningococcal CU-385 strain.

6. A vaccine for the treatment of neisserial, preferably meningococcal, disease comprising the multivalent meningococcal bleb composition of claim 1, and a pharmaceutically acceptable excipient.

7. The vaccine of claim 6 additionally comprising one or more plain or conjugated meningococcal capsular polysaccharides selected from the following list of serotypes: A, C, Y and W.

8. The vaccine of claim 6 suitable for use in New Zealand or Europe wherein the bleb preparation that is not deficient in PorA is derived from a meningococcal strain with a serosubtype of P1.4.

9. The vaccine of claim 6 suitable for use in USA where the bleb preparation that is not deficient in PorA is derived from a meningococcal strain with a serosubtype of P1.7,16.

10. The vaccine of claim 6 suitable for use in Norway wherein the bleb preparation that is not deficient in PorA is derived from a meningococcal strain with a serosubtype of P1.16.

11. A method of manufacturing the multivalent meningococcal bleb composition of claim 1 comprising the step of combining the bleb preparation that is not deficient in PorA with the bleb preparation that is deficient in PorA.

12. A method of preventing or treating neisserial, preferably meningococcal, disease comprising the step of administering an immunologically effective amount of the vaccine of claim 6 to a host in need thereof.

13. he use of an immunologically effective amount of the vaccine of claim 6 in the manufacuture of a medicament for the prevention or treatment of neisserial, preferably meningococcal, disease.

14. A method of manufacturing the vaccine of claim 6 comprising the step of combining the bleb preparation that is not deficient in PorA with the bleb preparation that is deficient in PorA.
